(19) United States
(12) Patent Application Publication
(10) Pub. No.: US 2024/0416344 A1
(43) Pub. Date: Dec. 19, 2024

(54) FLUID DEVICE, FLUID DELIVERY SYSTEM, AND TREATMENT METHOD OF FLUID CONTAINING METAL NANOPARTICLES

(71) Applicant: Hitachi, Ltd., Tokyo (JP)

(72) Inventors: Saaya SHIMIZU, Tokyo (JP); Tomoyuki SAKAI, Tokyo (JP); Yoshimitsu YANAGAWA, Tokyo (JP)

(21) Appl. No.: 18/583,000

(22) Filed: Feb. 21, 2024

(30) Foreign Application Priority Data
Jun. 15, 2023 (JP) 2023-098550

(51) Int. Cl. B01L 3/00; B01L 7/00; C12Q 1/6813; C12Q 1/6844; C12Q 1/6876

(52) U.S. Cl. CPC B01L 3/502761; B01L 7/52; C12Q 1/6813; C12Q 1/6844; C12Q 1/6876; B01L 2300/047; B01L 2300/0663; B01L 2300/0681; B01L 2300/0816

(57) ABSTRACT

Separation of metal nanoparticles and detection of a gene in a fluid from which the metal nanoparticles are separated are performed in different spaces. A fluid device includes: a gene amplification section that stores a fluid containing metal nanoparticles and that heats the fluid using a photothermal effect of the metal nanoparticles to amplify a gene in the fluid; a metal nanoparticles separating section that separates the metal nanoparticles from the fluid; and a gene detection section that is a space different from the metal nanoparticles separating section and that detects the gene in the fluid from which the metal nanoparticles are separated in the metal nanoparticles separating section.

### CROSS REFERENCE TO RELATED APPLICATIONS

The present application claims priority from Japanese patent application JP 2023-098550 filed on Jun. 15, 2023, the entire content of which is hereby incorporated by reference into this application.

### BACKGROUND

### Technical Field

The present disclosure relates to a fluid device, a fluid delivery system, and a treatment method of a fluid containing metal nanoparticles.

### Background Art

Testing for infections such as a respiratory infection and a bloodstream infection requires earlier start of appropriate treatment and quick pathogen detection to prevent secondary infections. Further, since various pathogens cause infections, it is important to simultaneously test a plurality of pathogens. The genetic testing based on a PCR (Polymerase Chain Reaction) method is superior in speed as compared to the conventional testing based on the microbial culturing, and has been actively studied in recent years.

The typical PCR amplifies a target DNA such that a micro tube containing a reaction solution is set in a heat block and the temperature of the heat block is changed to repeat the steps of denaturation, annealing, and extension (thermal cycle). At this time, the time of heating/cooling the heat block, not of the solution, is the rate-determining factor, and the reaction typically requires about one hour.

As an even quicker PCR, a technique (plasmonic PCR) using surface plasmon resonance of metal nanoparticles has been developed. When metal nanoparticles dispersed in a PCR solution are irradiated with light having a specific wavelength, the metal nanoparticles each exhibit surface plasmon resonance and the light energy is converted into the heat energy so that the solution around the metal nanoparticles is heated. Since metal nanoparticles as a heat source are dispersed in the solution, the solution can be heated more uniformly and quickly as compared to the conventional method of externally heating the solution using a heater. The heating rate by the metal nanoparticles is several ten to several hundred times the conventional method and the plasmonic PCR completes gene amplification only by several minutes.

Techniques of multiplex gene detection include a technique of identifying the types of genes using position information. For example, a microarray has probes fixed at specific positions on a small substrate and it is possible to detect genes using a hybridization reaction between the probes and the genes in a sample. With the position information of the probes, over hundreds of thousands of genes can be identified.

Another example of the multiplex gene detection using the position information is a real time PCR. In the real time PCR, it is common to modify a probe to be bound to a specific gene with different fluorescent dyes so as to identify the type of the gene from the detected wavelength of the fluorescent signal. The number of items for the identification using the fluorescence is limited by the separation capability of a detector, and is currently limited to around four items.

Thus, for a larger number of genes to be simultaneously tested, a sample is divided into a plurality of wells and the genes are identified using the position information of the wells. In this case, the number of items simultaneously tested is a product of the numbers of types of dyes and wells, and a larger number of genes can be simultaneously tested.

Further, in recent years, a technique has been developed in which a chemical reaction, such as nucleic acid amplification and gene detection, is performed in a flow path formed in a small substrate (fluid device). Since the chemical reaction is performed in a micro space, the use amount of a reagent can be reduced. Moreover, there are advantages in that contamination risk can be reduced because the reaction is performed in a closed flow path and testing can be performed at low cost because most fluid devices can be mass-produced.

### SUMMARY

When plasmonic PCR and gene detection using metal nanoparticles are performed in a fluid device, there is a problem in that the gene detection is inhibited by the metal nanoparticles. For example, when the gene detection is based on a fluorescent signal, it has been known that an excitation light with which a gene detection section is irradiated for signal detection is absorbed by the metal nanoparticles. Thus, the fluorescent signal to be obtained is reduced (Nicole R., et al., Nat. Nanotechnol., 17, 984-992, 2022). The reduction in the fluorescent signal decreases the sensitivity of the genetic testing, which could cause false negative. Further, it is conceivable that a micro flow path in the fluid device is clogged by the metal nanoparticles and whereby the fluid delivery rate is lowered to thus extend the testing time.

As a method of removing the metal nanoparticles used for the plasmonic PCR, Jiyong C., et al., “Nat. Biomed. Eng., 4, 1159-1167, 2020” discloses a method using metal nanoparticles having a magnetic substance as a core. The plasmonic PCR is performed in a microtube, using the nanoparticles of the magnetic substance coated with gold and the nanoparticles are caused to aggregate on the bottom surface of the microtube by a magnet. When the microtube is irradiated with an excitation light, a gene-derived fluorescent signal is obtained from portions other than the bottom surface of the microtube. In Jiyong C., et al., the plasmonic PCR, removal of nanoparticles, and gene detection are performed in the same site. In this technique, identification information of the genes based on the position information cannot be obtained. Further, the number of items to be simultaneously tested is limited to around four of those identifiable with the fluorescence. Meanwhile, about 30 items and about 5or more items are required to be simultaneously tested for the genetic testing for the bloodstream infection and for the respiratory infection, respectively.

Thus, the present disclosure provides a fluid device, a fluid delivery system, and a treatment method of a fluid containing metal nanoparticles capable of multiplex gene detection by performing, in different spaces, separation of metal nanoparticles and detection of genes in the fluid from which the metal nanoparticles are separated.

A fluid device of the present disclosure includes a gene amplification section that stores a fluid containing metal nanoparticles, the gene amplification section configured to heat the fluid using a photothermal effect of the metal nanoparticles to amplify a gene in the fluid; a metal nanoparticles separating section configured to separate the metal nanoparticles from the fluid; and a gene detection section that is a space different from the metal nanoparticles separating section, the gene detection section configured to detect the gene in the fluid from which the metal nanoparticles are separated in the metal nanoparticles separating section.

A fluid delivery system of the present disclosure includes: a fluid device including a gene amplification section that stores a fluid containing metal nanoparticles, the gene amplification section configured to heat the fluid using a photothermal effect of the metal nanoparticles to amplify a gene in the fluid, a metal nanoparticles separating section configured to separate the metal nanoparticles from the fluid, and a gene detection section that is a space different from the metal nanoparticles separating section, the gene detection section configured to detect the gene in the fluid from which the metal nanoparticles are separated in the metal nanoparticles separating section; and a fluid delivery apparatus in which the fluid device can be installed, the fluid delivery apparatus configured to deliver, from the metal nanoparticles separating section to the gene detection section, the fluid from which the metal nanoparticles are separated in the metal nanoparticles separating section.

A treatment method of a fluid containing metal nanoparticles of the present disclosure includes: heating a fluid using a photothermal effect of metal nanoparticles contained in the fluid to amplify a gene in the fluid; separating the metal nanoparticles from the fluid; delivering, from a first space where the metal nanoparticles are separated to a second space different from the first space, the fluid from which the metal nanoparticles are separated; and detecting the gene in the fluid in the second space.

According to the present disclosure, multiplex gene detection is possible by performing, in different spaces, separation of metal nanoparticles and detection of genes in the fluid from which the metal nanoparticles are separated. The issues, configurations, and effects other than the aforementioned matters will be apparent from the description of the following embodiments.

### BRIEF DESCRIPTION OF THE DRAWINGS

FIG. 1 is a configurational view of a fluid device of Example 1;

FIG. 2 is a cross-sectional view of the fluid device of Example 1;

FIG. 3A is an apparatus configurational view of a fluid delivery system of Example 1;

FIG. 3B is a hardware block diagram of a control section of Example 1;

FIG. 4 is an operation flow of Example 1;

FIG. 5 is a schematic view of the fluid device in step (c) of FIG. 4;

FIG. 6 is a schematic view of the fluid device in step (e) of FIG. 4;

FIG. 7 is a schematic view of the fluid device in step (f) of FIG. 4;

FIG. 8 is a configurational view of the fluid device of a modification 1 of Example 1;

FIG. 9 is a configurational view of the fluid device of the modification 1 of Example 1;

FIG. 10 is an apparatus configurational view of the fluid delivery system of the modification 1 of Example 1;

FIG. 11 is a configurational view of the fluid device of a modification 2 of Example 1;

FIG. 12 is a cross-sectional view of the fluid device of the modification 2 of Example 1;

FIG. 13 an apparatus configurational view of the fluid delivery system of the modification 2 of Example 1;

FIG. 14 is a configurational view of the fluid device of Example 2;

FIG. 15 is a cross-sectional view of the fluid device of Example 2;

FIG. 16 is an apparatus configurational view of the fluid delivery system of Example 2;

FIG. 17 is an operation flow of Example 2;

FIG. 18 is a schematic view of the fluid device in step (d) of FIG. 17;

FIG. 19 is a schematic view of the fluid device in step (e) of FIG. 17;

FIG. 20 is a configurational view of the fluid device of Example 3;

FIG. 21 is a cross-sectional view of the fluid device of Example 3;

FIG. 22 is an apparatus configurational view of the fluid delivery system of Example 3;

FIG. 23 is an operation flow of Example 3;

FIG. 24 is a schematic view of the fluid device in step (d) of FIG. 23;

FIG. 25 is a schematic view of the fluid device in step (e) of FIG. 23;

FIG. 26 is a configurational view of the fluid device of Example 4;

FIG. 27 is a cross-sectional view of the fluid device of Example 4;

FIG. 28 is an apparatus configurational view of the fluid delivery system of Example 4;

FIG. 29 is an operation flow of Example 4;

FIG. 30 is a schematic view of the fluid device in step (d) of FIG. 29; and

FIG. 31 is a schematic view of the fluid device in step (e) of FIG. 29.

### DETAILED DESCRIPTION

Embodiments of the present disclosure will be described in detail based on the drawings. In the following embodiments, it goes without saying that the constituent elements (also including elemental steps and the like) are not necessarily essential, unless otherwise particularly explicitly indicated, and excluding cases in which the constituent elements are considered principally, apparently essential.

### Example 1

Hereinafter, Example 1 will be described. The present example relates to a fluid device and a fluid delivery apparatus for performing gene amplification using metal nanoparticles, removal of metal nanoparticles using a magnetic force, and multiplex gene detection based on the chromatography method.

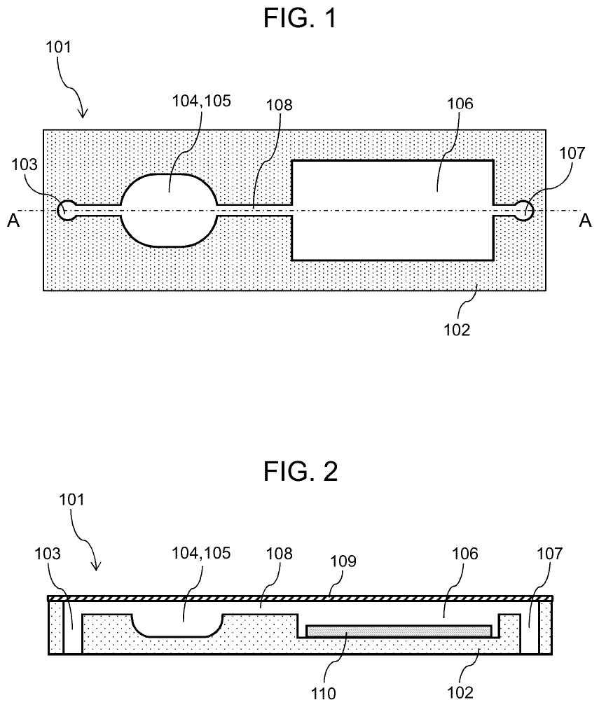

FIG. 1 illustrates a plan view of a fluid device 101. FIG. 2 illustrates a cross- sectional view taken along line A-A of FIG. 1. The fluid device 101 includes a substrate 102, a sealing material 109, and a solid phase carrier section 110.

The substrate 102 is preferably formed of a material that is stable against change in temperature and pressure, is not easily affected by a solution used for a chemical reaction, and is easy to form. As such a material, for example, cyclo olefin polymer (COP), polycarbonate (PC), and acrylic resin (PMMA) are preferable.

The substrate 102 includes a solution introducing section 103, a gene amplification section 104, a metal nanoparticles separating section 105, a gene detection section 106, a valve connecting section 107, and a flow path 108. The gene amplification section 104 can store a fluid (solution) containing metal nanoparticles. The gene amplification section 104 heats the fluid using the photothermal effect using surface plasmon resonance of metal nanoparticles to amplify a gene in a fluid. The metal nanoparticles separating section 105 separates the metal nanoparticles from the fluid. The gene detection section 106 is a space different from the metal nanoparticles separating section 105 and detects the gene in the fluid from which the metal nanoparticles are separated in the metal nanoparticles separating section 105.

The metal nanoparticles separating section 105 and the gene detection section 106 are connected by means of the flow path 108.

The solution introducing section 103 and the valve connecting section 107 are processed as through-holes on the substrate 102 and open on a surface opposite to a surface where a sealing material 109 is crimped. The gene amplification section 104, the metal nanoparticles separating section 105, the gene detection section 106, and the flow path 108 are formed such that the substrate 102 is processed by cutting and sealed with the sealing material 109.

In the present example, as illustrated in FIG. 1 and FIG. 2, the gene amplification section 104 and the metal nanoparticles separating section 105 are positioned in the same space on the substrate 102. Alternatively, the gene amplification section 104 and the metal nanoparticles separating section 105 may be disposed in different spaces on the substrate 102. In this case, the gene amplification section 104 and the metal nanoparticles separating section 105 need to be connected by means of a flow path.

The gene amplification section 104 and the metal nanoparticles separating section 105 are preferably in a shape without corners to minimize a remaining sample solution. An example of the preferable shape of the gene amplification section 104 and the metal nanoparticles separating section 105 is oval illustrated in FIG. 1 and FIG. 2, but not limited thereto, and may be circular. The gene amplification section 104 and the metal nanoparticles separating section 105 may also be in a flow-path shape such as the flow path 108 illustrated in FIG. 1 and FIG. 2.

The gene detection section 106 does not need to be in a rectangular parallelepiped as illustrated in FIG. 1 and FIG. 2, and may be, for example, in a columnar shape. The gene detection section 106 may also be in a flow-path shape such as the flow path 108 of FIG. 1 and FIG. 2.

The flow path 108 does not need to be in a linear shape as illustrated in FIG. 1 and FIG. 2, but may be, for example, in a meandering shape. In the example of FIG. 1, the flow path 108 is one flow path with no branches, but may be a flow path with a plurality of branches. The cross-sectional shape of the flow path 108 can be designed in any shape, for example, in a square, rectangular, or circular shape.

The sealing material 109 has a feature of adhering to the substrate 102. The surface of the sealing material 109 adhering to the substrate 102 may be adhesive or pressure sensitive. The sealing material 109 is preferably made of polyolefin (PO) or polypropylene (PP). The sealing material 109 is preferably a resin film having a thickness of around 0.1 mm.

The solid phase carrier section 110 is fixed to the fluid device 101 for performing multiplex gene detection based on the chromatography method. Several types of specific molecules to be chemically reacted with genes of a test subject are fixed to the solid phase carrier section 110 based on the position information. Examples of the specific molecules include a nucleic acid, an antigen, an antibody, and a peptide.

For the solid phase carrier section 110, any known shape and material, without being particularly limited, can be adopted, as long as the form allows a solution to be diffused by capillarity. Examples of the solid phase carrier section 110 include a filter paper in a sheet or a rod shape, silica gel, polyether sulfone, and nitrocellulose.

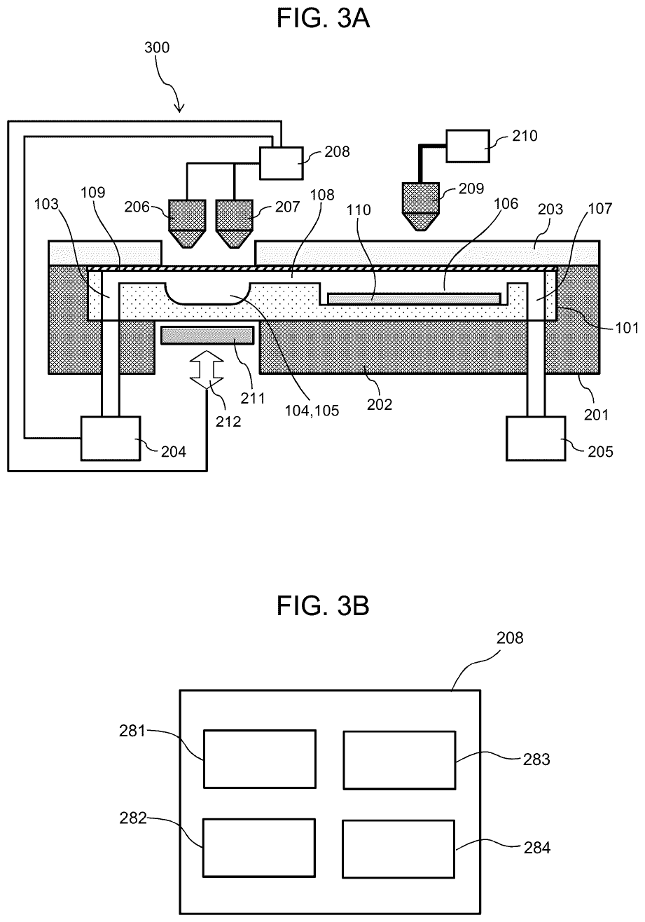

Next, description will be made of the fluid delivery apparatus in which any of the fluid device of FIG. 1 and FIG. 2 and the modifications can be installed. As an example, FIG. 3A illustrates a fluid delivery apparatus 201 (fluid delivery system 300) in which the fluid device of FIG. 1 and FIG. 2 is installed. The fluid delivery system 300 includes the fluid device 101 and the fluid delivery apparatus 201. The delivery device 201 can be used to deliver a solution in the fluid device 101. Further, the fluid delivery apparatus 201 can provide, to the fluid device 101, light, air pressure, a sensor, and the like that are necessary for performing gene amplification using metal nanoparticles, removal of the metal nanoparticles using a magnetic force, and the multiplex gene detection in the fluid device 101.

The fluid delivery apparatus 201 includes a device retaining section 202, a cover 203, a pressurizing/depressurizing device 204, a valve 205, a temperature sensor 206, a light source 207, a control section 208, a sensor 209, a signal detection section 210, and an external magnetic source 211.

For the device retaining section 202, it is preferable to adopt a material having a heat resistance and a low thermal conductivity. For example, PEEK (polyether ether ketone) and PC (polycarbonate) are preferable.

The cover 203 functions to fix, to a predetermined position, the fluid device 101 installed in the device retaining section 202. For the cover 203, it is preferable to adopt a material having a heat resistance and a low thermal conductivity. For example, PC (polycarbonate) can be used.

The pressurizing/depressurizing device 204 is an example of a fluid delivery means, and is connected to the solution introducing section 103. The pressurizing/depressurizing device 204 pressurizes or depressurizes the inside of the fluid device 101 so as to deliver the solution via the solution introducing section 103.

The valve 205 is, for example, an on-off valve. The valve 205 may be a directional valve or may be a directional valve provided in a tube that is connected to the valve connecting section 107. The valve 205 may also be a microvalve that can be installed inside the fluid device 101.

The temperature sensor 206 is used to measure the temperature of the solution in the gene amplification section 104. The temperature sensor 206 is, for example, a noncontracting infrared sensor. The temperature sensor 206 may be a thermocouple contacting the solution in the gene amplification section 104.

The light source 207 is used to irradiate the solution in the gene amplification section 104 with light having a specific wavelength so as to induce plasmon resonance of the metal nanoparticles in the solution. The light source 207 is, for example, an LED or a laser. For the specific wavelength, a wavelength close to the absorption maximum (reflection minimum) wavelength regarding the surface plasmon resonance of the metal nanoparticles used is selected. The absorption maximum (reflection minimum) wavelength regarding the surface plasmon resonance of the metal nanoparticles is present, for example, between 500 nm and 1400 nm, and changes depending on the composition, shape, or size of the metal nanoparticles. In FIG. 3A, to improve the irradiation efficiency by the light source 207, the cover 203 in a region between the light source 207 and the gene amplification section 104 opens. However, the shape of the cover 203 is not limited thereto. The cover 203 may not open.

The control section 208 is used to control the temperature sensor 206 and the light source 207. The control section 208 controls the temperature sensor 206 and the light source 207 so that the solution in the gene amplification section 104 can be controlled at a desired temperature. An example of the control of the solution temperature in the gene amplification section 104 by the control section 208 is as follows.

When the light source 207 irradiates the solution in the gene amplification section 104 with light under the control of the control section 208, the light energy is converted into the heat energy by surface plasmon resonance of the metal nanoparticles so that the solution is heated. The control section 208 acquires the solution temperature measured by the temperature sensor 206. When the control section 208 determines that the solution temperature has risen to a target temperature, the control section 208 stops light irradiation by the light source 207. When the control section 208 determines that the solution temperature has lowered to the next target temperature, the control section 208 resumes the light irradiation by the light source 207 to reheat the solution. By repeating the aforementioned steps a predetermined number of times, the thermal cycle of the PCR (Polymerase Chain Reaction) is realized.

Here, with reference to FIG. 3B, the hardware configuration of the control section 208 will be described. The control section 208 includes a processor 281, a primary memory section 282, an auxiliary memory section 283, and an interface 284. The processor 281 is a CPU (Central Processing Section), a GPU (Graphics Processing Section), a DSP (Digital Signal Processor), an ASIC, or the like. The primary memory section 282 is a DRAM (Dynamic Random Access Memory) or the like, and is used as a work area for the processor 281. The auxiliary memory section 283 is an HDD (Hard Disk Drive), an SSD (Solid State Drive), a combination thereof, or the like, and stores various programs and data. The interface 284 is a device controller or the like that controls the operations of the peripheral equipment (the pressurizing/depressurizing device 204, a moving mechanism 212, the temperature sensor 206, and the light source 207) connected to the control section 208.

For example, the auxiliary memory section 283 stores a temperature control program for implementing the aforementioned thermal cycle of the PCR. The processor 281 executes the temperature control program to control the light irradiation by the light source 207 and the stop of the light irradiation while monitoring the temperature information output from the temperature sensor 206.

In addition, the auxiliary memory section 283 stores a treatment control program for treating the fluid in the fluid device 101. The processor 281 executes the treatment control program to control the operation of the pressurizing/depressurizing device 204 so as to deliver the fluid containing metal nanoparticles from the metal nanoparticles separating section 105 to the gene detection section 106 via the flow path 108. Further, the processor 281 controls the operation of the moving mechanism 212 described later to bring the external magnetic source 211 into close contact with the metal nanoparticles separating section 105 when separating the metal nanoparticles. Furthermore, the processor 281 controls the operation of the moving mechanism 212 described later to keep the external magnetic source 211 close contact with the metal nanoparticles separating section 105 when delivering the fluid.

When the external magnetic source 211 is an electromagnet, the processor 281 (energization control section) controls the energization of the external magnetic source 211 (electromagnet).

The sensor 209 acquires information to observe the gene detection reaction in the gene detection section 106. The signal obtained by the sensor 209 is transmitted to the signal detection section 210. For the sensor 209, at least one of a fluid level detection sensor, a fluorescent detection sensor, and an image recognition sensor can be used. For example, when the fluid level detection sensor is used, the position of the solution in the solid phase carrier section 110 can be more properly controlled. When the image recognition sensor is used, it is possible to acquire the position information of the genes detected by the solid phase carrier section 110 and the types of the genes can be identified. To improve the observation efficiency of the sensor 209, the cover 203 in a region between the sensor 209 and the gene detection section 106 may open.

The external magnetic source 211 is used to remove, by a magnetic force, the metal nanoparticles containing a magnetic substance contained in the solution in the metal nanoparticles separating section 105. The external magnetic source 211 is, for example, a magnet, such as a neodymium magnet and a ferrite magnet. When a magnet is used as the external magnetic source 211, as illustrated in FIG. 3A, it is preferable to provide the moving mechanism 212 that changes the distance between the gene amplification section 104 or the metal nanoparticles separating section 105 and the external magnetic source 211. In this manner, during the plasmonic PCR, it is possible to keep the external magnetic source 211 away from the gene amplification section 104. By keeping the external magnetic source 211 away from the gene amplification section 104, the metal nanoparticles are dispersed in the PCR solution so that the solution can be uniformly heated by the surface plasmon resonance of the metal nanoparticles.

The external magnetic source 211 may be, for example, an electromagnet. When the electromagnet is used as the external magnetic source 211, by starting energization of the electromagnet after completing the plasmonic PCR, the metal nanoparticles can be remained dispersed during the plasmonic PCR. This eliminates a drive mechanism for a magnet, thereby enabling to more simplify and downsize the fluid delivery apparatus 201.

Mirror finishing to reflect light having an irradiation wavelength band may be applied on the surface of the external magnetic source 211 or between the gene amplification section 104 and the electromagnet. Thus, the light permeated through the solution without being absorbed by the nanoparticles can be returned to the solution so as to be irradiated to the metal nanoparticles. As a result, it is possible to efficiently use the energy of the irradiated light to heat the solution.

The form of the fluid delivery apparatus 201 is not limited to that illustrated in FIG. 3A, and may vary depending on the shape of the fluid device 101, the gene detection technique in the gene detection section 106, and other circumferences. For example, in FIG. 3A, the temperature sensor 206, the light source 207, and the sensor 209 are disposed on the side of the sealing material 109 of the fluid device 101, but may be on the opposite side of the scaling material 109. In this case, it is preferable to cut any region of the device retaining section 202 so as not to interrupt the operations of the temperature sensor 206, the light source 207, and the sensor 209.

For example, the arrangement of the pressurizing/depressurizing device 204 and the valve 205 may be interchanged from the arrangement of FIG. 3A. A filter to prevent contamination of genes may be provided between the fluid device 101 and at least one of the pressurizing/depressurizing device 204 or the valve 205. The filter may be provided inside the fluid device 101.

The fluid delivery apparatus 201 may include a cooling mechanism to cool the gene amplification section 104. With the cooling mechanism for the gene amplification section 104, the cooling of the solution in the gene amplification is expedited so that the plasmonic PCR can be expedited. The cooling mechanism may be, for example, cooling performed using a fan. Alternatively, the cooling mechanism may be a method of bringing an external heat source into close contact with the gene amplification section 104.

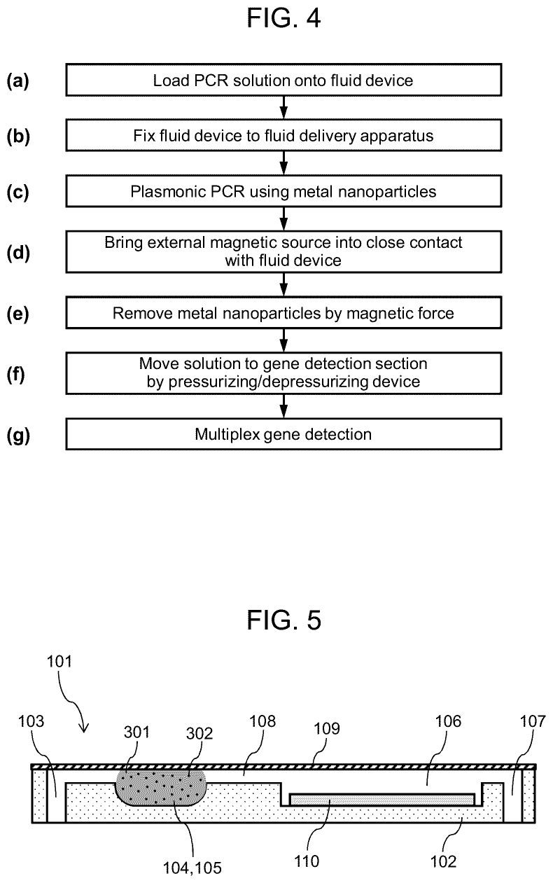

FIG. 4 illustrates an example of an operation flow of genetic testing using the fluid delivery apparatus 201. The operation flow is for performing the plasmonic PCR using metal nanoparticles, removal of the metal nanoparticles, and the multiplex gene detection. Hereinafter each step of the operation will be described following FIG. 4.

First, a solution used for the plasmonic PCR using metal nanoparticles is introduced from the solution introducing section 103 and is placed in the gene amplification section 104 (FIG. 4(a)). The method of introducing the solution to the fluid device 101 is not limited thereto. For example, when the fluid delivery apparatus 201 is a syringe pump, the introducing may be made such that the solution is introduced into the syringe and is delivered from the syringe to the fluid device.

The solution used in the present example is composed of, for example, a mixture of a sample solution as a test subject, one or more pairs of primers, a heat-resistant enzyme, four types of deoxyribonucleoside triphosphate (dATP, dCTP, dGTP, dTTP), and metal nanoparticles.

The metal nanoparticles used in the present example are removed by a magnetic force after the plasmonic PCR and thus, contain the magnetic substance. The metal nanoparticle containing the magnetic substance may be obtained, for example, such that the surrounding of the core of the magnetic substance is coated with a metal layer, or may be obtained such that material of a mixture of metal and the magnetic substance is formed as the nanoparticle.

The metal nanoparticle can be determined in any size and shape. For example, the longer side or the diameter of the metal nanoparticle is from 1 nm to 1000 nm, preferably, from 2 nm to 500 nm.

The metal nanoparticles used in the present example preferably contain gold. The metal nanoparticles may contain a Group 11 element such as silver and copper. Examples of the material of the metal nanoparticles that exhibits the surface plasmon resonance further include magnesium, aluminum, gallium, indium, ruthenium, or nickel. For use in the plasmonic PCR, selection needs to be made by considering inhibition of the gene amplification by these metals, the chemical resistance of the metals, and the like.

The metal nanoparticles used in the present example preferably undergo surface modification to prevent aggregation of particles so as to be dispersed in the solution to uniformly heat the solution. Methods of the surface modification include a method in which the surfaces of the metal nanoparticles are charged by such as silica coating and phosphine- sulfonate modification so that the particles are electrostatically repulsive each other.

Of the solution used in the present example, a part or all of the components other than the sample solution as a test subject may be retained, in a liquid or a solid state, in advance in the fluid device. For example, all of the components other than the sample solution may be retained as a freeze-dried reagent in the gene amplification section 104 in advance and the freeze-dried reagent may be dissolved by the sample solution.

Next, the fluid device 101 is fixed to the fluid delivery apparatus 201 (FIG. 4(b)). In FIG. 4, the PCR solution is introduced onto the fluid device 101 (FIG. 4(a)) and then, the fluid device 101 with the PCR solution introduced is fixed to the fluid delivery apparatus 201 (FIG. 4(b)). Alternatively, after the fluid device 101 is fixed (FIG. 4(b)), the PCR solution may be introduced onto the fixed fluid device 101 (FIG. 4(a)).

Subsequently, the gene amplification section 104 performs the plasmonic PCR using metal nanoparticles (FIG. 4(c)). FIG. 5 schematically illustrates the position of a solution 301 (solution used for the PCR other than the metal nanoparticles) and the state of the metal nanoparticles 302 at this stage. At the stage of FIG. 4(c), the magnetic force of the external magnetic source 211 does not act on the metal nanoparticles 302 and the metal nanoparticles 302 are dispersed in the solution 301.

As the plasmonic PCR, it is possible to adopt a method called two-step PCR in which, for example, PCR is performed by repeating denaturation, annealing, and extension in two temperature regions. The control section 208 alternately changes the temperature of the solution between the denaturation temperature (e.g., 95° C.) and the annealing and extension temperature (e.g., 60° C.) so that a desired thermal cycle can be achieved.

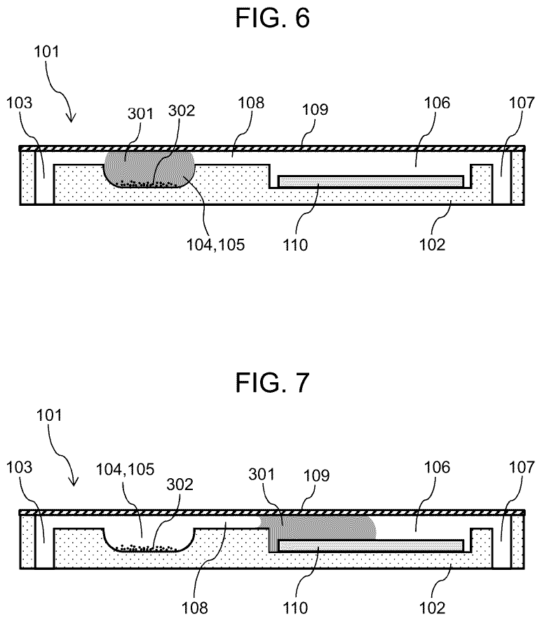

Next, the external magnetic source 211 (here, a magnet) is brought into close contact with the fluid device (FIG. 4(d)). In this manner, the magnetic force from the external magnetic source 211 is applied to the metal nanoparticles 302 so that the metal nanoparticles 302 aggregate on the bottom surface on the side of the external magnetic source 211 of the metal nanoparticles separating section 105 (FIG. 4(c)). FIG. 6 schematically illustrates the position of the solution 301 and the state of the metal nanoparticles 302 at this stage.

Next, the solution 301 is moved to the gene detection section 106 by means of the pressurizing/depressurizing device 204 (FIG. 4(f)). FIG. 7 schematically illustrates the position of the solution 301 and the state of the metal nanoparticles 302 at this stage. In the step of FIG. 4(f), the external magnetic source 211 is kept close contact with the fluid device 101. Thus, the solution 301 can be delivered to the gene detection section 106, with the metal nanoparticles 302 captured by the metal nanoparticles separating section 105.

Finally, the gene detection section 106 performs the multiplex gene detection (here, the nucleic acid chromatography) (FIG. 4(g)).

Note that in the present example, the double-stranded DNA amplified in the PCR may be provided for the gene detection after being resolved into the single-stranded DNA. For example, when the chromatography performed in the gene detection section 106 is based on a hybridization reaction among DNAs, the efficiency of the hybridization reaction is expected to be improved by resolving the double-stranded DNA into the single-stranded DNA. The method of resolving the double-stranded DNA into the single-stranded DNA is, for example, thermal denaturation. In addition, the examples of the method include a method using a nucleic acid denaturant, such as urea and formamide, and dissociation with a sodium hydroxide solution. A single-stranded DNA can also be obtained such that a double-stranded DNA modified, at its terminus of the strand on one side, with biotin is obtained in the PCR and the solution of the double-stranded DNA is contacted with beads or columns with their surfaces modified with avidin.

Further, enzymolysis with nuclease, such as Lambda Exonuclease, can also be used. In this case, the single-stranded DNA can be obtained by modifying the primer used in the PCR with a recognition site of nuclease.

Another method to improve the efficiency of the gene detection based on the hybridization reaction is a technique using a stop primer in performing the PCR. A primer that has a sequence (tag sequence) complementary to a probe used in the gene detection and that is modified to stop the DNA extension is used to perform the PCR to obtain a double-stranded DNA having, at its terminus, a single-stranded tag sequence. Since the hybridization reaction takes place between a probe and a single-stranded tag sequence, the efficiency of the gene detection can be improved.

The configurations of the fluid device, the fluid delivery apparatus, and the operation flow when the chromatography method is adopted have been described so far, as the example of the multiplex gene detection technique.

### Modification 1 of Example 1

Next, as a modification of Example 1, the configuration of the fluid device and the fluid delivery apparatus that perform the multiplex gene detection using a method other than the chromatography method will be described. Note that the operation flow is not illustrated, since the content of FIG. 4(g) is the only difference. Further, the descriptions of the portions common to FIG. 1 to FIG. 3B will be omitted in some cases.

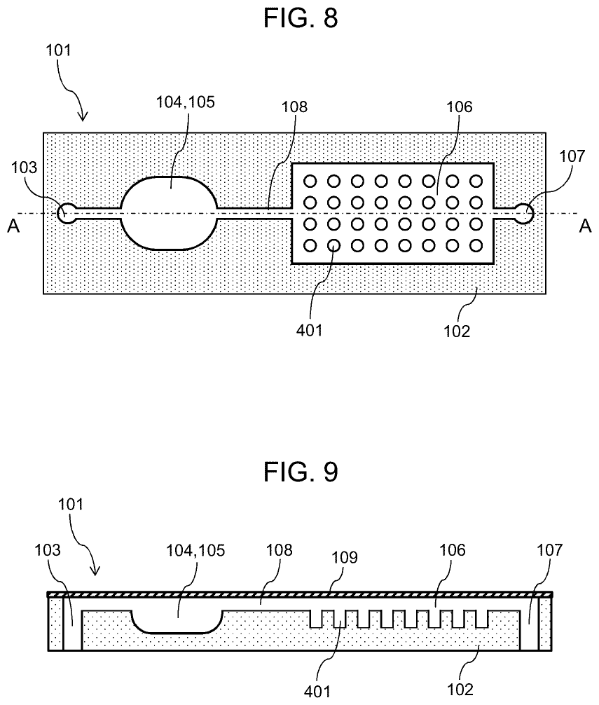

Another example of the multiplex gene detection technique is a method of performing the real time PCR using a plurality of wells having position information, for example. FIG. 8 illustrates a plan view of the fluid device 101 that is a component in which the gene detection section 106 performs the real time PCR. FIG. 9 illustrates a cross-sectional view taken along line A-A of FIG. 8.

The fluid device 101 illustrated in FIG. 8 and FIG. 9 includes a plurality of wells 401 that are assigned position information in the gene detection section 106. Each well 401 retains, in advance, the PCR primer in a solid or a liquid form for amplifying the specific gene corresponding to the position information. In addition, each well 401 can also retain, in advance, a reagent in a solid or a liquid form other than the primer used for the PCR.

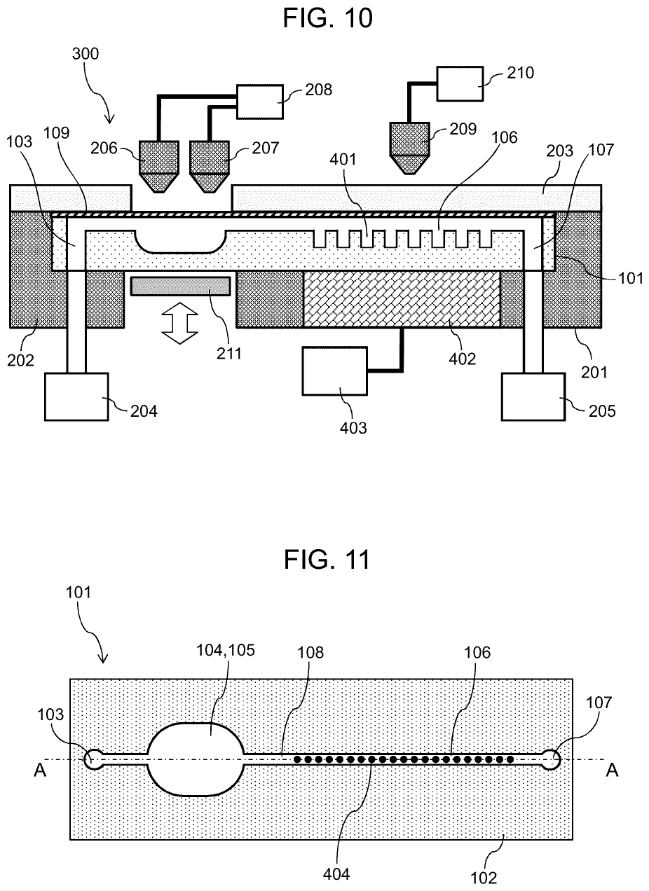

FIG. 10 illustrates the fluid delivery apparatus 201 with the fluid device of FIG. 8 and FIG. 9 installed. The fluid delivery apparatus 201 of FIG. 10 includes an external heat source 402 and a temperature control section 403 in addition to the fluid delivery apparatus 201 of FIG. 3A. The external heat source 402 includes a built-in thermocouple and heater and is maintained at a desired temperature by the temperature control section 403. The gene detection section 106 is heated by the external heat source 402. With the control by the temperature control section 403, the thermal cycle in the wells 401 in the gene detection section 106 is achieved so that the multiplex gene detection based on the real time PCR is performed.

Note that the hardware configuration of the temperature control section 403 is the same as that of the control section 208 illustrated in FIG. 3B and thus, the description will be omitted. Note that the form of the fluid delivery apparatus 201 is not limited to that illustrated in FIG. 10. For example, in FIG. 10, the temperature sensor 206, the light source 207, and the sensor 209 are disposed on the side of the sealing material 109 of the fluid device 101, but may be on the opposite side of the sealing material 109. In FIG. 10, the external heat source 402 is disposed on the opposite side of the sealing material 109 of the fluid device 101, but may be on the side of the sealing material 109.

For example, the arrangement of the pressurizing/depressurizing device 204 and the valve 205 may be interchanged from the arrangement of FIG. 10. A filter to prevent contamination of genes may be provided between the fluid device 101 and at least one of the pressurizing/depressurizing device 204 or the valve 205. In addition, the fluid delivery apparatus 201 may include a cooling mechanism for the gene amplification section 104.

### Modification 2 of Example 1

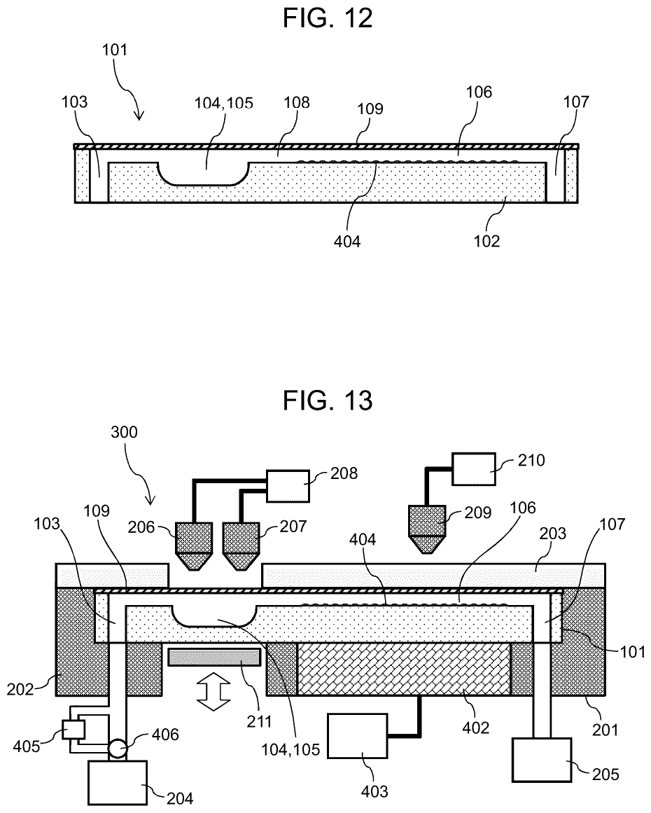

Further another example of the multiplex gene detection technique is a technique based on a microarray method. FIG. 11 illustrates a plan view of the fluid device 101 that is a component in which the gene detection section 106 performs the gene detection based on the microarray method. FIG. 12 illustrates a cross-sectional view taken along line A-A of FIG. 11.

The fluid device 101 illustrated in FIG. 11 and FIG. 12 includes a plurality of probe spots 404 that are assigned position information in the gene detection section 106. A probe corresponding to a gene as a detection target is fixed to the probe spot 404. The gene detection section 106 is not limited to a single linear flow path without having branches as illustrated in FIG. 11 and FIG. 12, and may be in a meandering shape or may have a plurality of branches, for example. The gene detection section 106 is not limited to be in a flow path shape as illustrated in FIG. 11 and FIG. 12. The gene detection section 106 may be, for example, in a rectangular parallelepiped as illustrated in FIG. 1 and FIG. 2, or the probe spots 404 may be two-dimensionally arranged in the gene detection section 106 in such a shape.

FIG. 13 illustrates the fluid delivery apparatus 201 with the fluid device 101 of FIG. 11 and FIG. 12 installed. The fluid delivery apparatus 201 of FIG. 13 includes the external heat source 402, the temperature control section 403, a cleaning solution storage 405, and a directional valve 406 in addition to the fluid delivery apparatus 201 of FIG. 1.

The external heat source 402 includes a built-in thermocouple and heater and is maintained at a predetermined temperature by the temperature control section 403. The gene detection section 106 is heated by the external heat source 402. With the control by the temperature control section 403, the gene detection section 106 is heated to a temperature (e.g., 45° C., 50° C., 55° C., or 60° C.) suitable for the hybridization reaction so that the multiplex gene detection based on the microarray method is performed.

The cleaning solution storage 405 and the directional valve 406 are provided to be used for a cleaning step to suppress the background light in performing the multiplex gene detection. In the case of the gene detection technique based on a fluorescent signal as in the microarray method, the background light can be reduced by performing the cleaning step after the gene detection reaction. In the case of the present example, the cleaning step is realized with the following operation.

After the hybridization reaction, the solution used for the hybridization reaction is discharged from the gene detection section 106. As the discharging method, there is a method of discharging the solution to the outside of the fluid delivery apparatus 201 through the valve 205. The solution may also be discharged to the outside of the fluid delivery apparatus 201 through the pressurizing/depressurizing device 204. Moreover, a chamber for a waste fluid may be provided in the fluid device 101 to which the solution is discharged. In doing so, the gene amplification section 104 or the metal nanoparticles separating section 105 may also serve as the chamber for the waste fluid.

Next, by switching the directional valve 406, the pressurizing/depressurizing device 204 and the cleaning solution storage 405 are connected. The cleaning step is performed such that the pressurizing/depressurizing device 204 is operated to deliver a cleaning solution in the cleaning solution storage 405 to the gene detection section 106. The position of the cleaning solution storage 405 is not limited to that illustrated in FIG. 13, and may be, for example, inside the fluid device 101.

Note that the form of the fluid delivery apparatus 201 is not limited to that illustrated in FIG. 13. For example, in FIG. 13, the temperature sensor 206, the light source 207, and the sensor 209 are disposed on the side of the sealing material 109 of the fluid device 101, but may be on the opposite side of the sealing material 109. In FIG. 13, the external heat source 402 is disposed on the opposite side of the sealing material 109 of the fluid device 101, but may be on the side of the sealing material 109.

For example, the arrangement of the pressurizing/depressurizing device 204 and the valve 205 may be interchanged from the arrangement of FIG. 13. A filter to prevent contamination of genes may be provided between the fluid device 101 and at least one of the pressurizing/depressurizing device 204 or the valve 205. In addition, the fluid delivery apparatus 201 may include a cooling mechanism for the gene amplification section 104.

Note that the double-stranded DNA amplified in the PCR may be provided for the hybridization reaction after being resolved into the single-stranded DNA. The resolving into the single-stranded DNA may be performed either before or after the removal of the metal nanoparticles.

### Modification 3 of Example 1

Further another example of the multiplex gene detection technique is a technique based on the beads array method. In this case, the gene detection section 106 is provided with a plurality of types of beads, on the surfaces of which the probes are fixed. The beads may be two-dimensionally arranged on the substrate 102 of the gene detection section 106 in a rectangular parallelepiped or columnar shape. The beads may also be linearly arranged on the gene detection section 106 in a shape of one flow path or a plurality of flow paths. In the multiplex gene detection based on the beads array method, genes may be identified by the arranged order of the beads. Furthermore, genes may be identified by labeling the beads with different fluorescence or the like.

### Modification 4 of Example 1

Further another example of the multiplex gene detection technique is a technique based on an electrophoresis method. In this case, the gene detection section 106 is provided with an electrophoretic carrier and an electrolyte. The electrophoretic carrier may be a capillary having, on its inner wall, an ionic functional group such as silica, or a substance having a molecular sieve effect, such as a polyacrylamide gel or an agarose gel. For the electrolyte, any of those known may be adopted without being particularly limited, as long as the electrolyte is an ion solution having an electro-conductivity. In the multiplex gene detection based on the electrophoresis method, genes may be identified by the electrophoresis distance. Genes may also be identified by the electrophoresis time.

### Treatment Method of Fluid Containing Metal Nanoparticles

Here, a treatment method of a solution (fluid containing metal nanoparticles) will be described. The treatment method of a solution includes: heating a solution using a photothermal effect of metal nanoparticles contained in the solution (performing the thermal cycle of the solution) to amplify a gene in the solution; separating the metal nanoparticles from the solution using a magnetic force (magnet 211 or electromagnet), a filter 501, a buffer 601, a gold thin film 701, and the like; delivering, from a first space (metal nanoparticles separating section 105) where the metal nanoparticles are separated to a second space (gene detection section 106) different from the first space, the solution from which the metal nanoparticles are separated; and detecting the gene in the fluid in the second space. For example, the aforementioned amplifying the gene, separating the metal nanoparticles, and delivering the solution are performed with the control by the control section 208. The detecting the gene is performed by the signal detection section 210.

The aforementioned separating the metal nanoparticles includes: separating the metal nanoparticles containing the magnetic substance from the solution by bringing a magnet (external magnetic source 211) into close contact with the first space (metal nanoparticles separating section 105); or separating the metal nanoparticles containing the magnetic substance from the solution by energizing an electromagnet (external magnetic source 211) provided near the first space.

The delivering, to the second space, the solution from which the metal nanoparticles are separated, includes delivering, to the second space (gene detection section 106), the solution from which the metal nanoparticles are separated, with the magnet (external magnetic source 211) kept close contact with the first space (metal nanoparticles separating section 105) or with the electromagnet (external magnetic source 211) energized.

### Advantageous Effect of Example 1

According to the fluid device 101 of Example 1, the plasmonic PCR using metal nanoparticles can be performed in the fluid device 101. Further, the gene amplified and the metal nanoparticles can be separated in the metal nanoparticles separating section 105 and the solution from which the metal nanoparticles are separated can be delivered to the gene detection section 106. In Example 1, it is possible to perform the multiplex gene detection based on the position information in the gene detection section 106 that is another space where the metal nanoparticles have been separated. Therefore, quick and multiplex genetic testing can be performed with no inhibition of the gene detection by the metal nanoparticles.

The metal nanoparticles separating section 105 and the gene detection section 106 are connected by means of the flow path, so that the separation of the metal nanoparticles and the gene detection can be performed in different spaces in the same device.

The gene amplification section 104 and the metal nanoparticles separating section 105 are in the same space, so that the fluid device 101 can be downsized.

### Example 2

Hereinafter, Example 2 will be described. The present example relates to the fluid device and the fluid delivery apparatus for performing gene amplification using metal nanoparticles, removal of metal nanoparticles using a filter, and multiplex gene detection based on the chromatography method. Note that the descriptions of the portions common to Example 1 will be omitted in some cases.

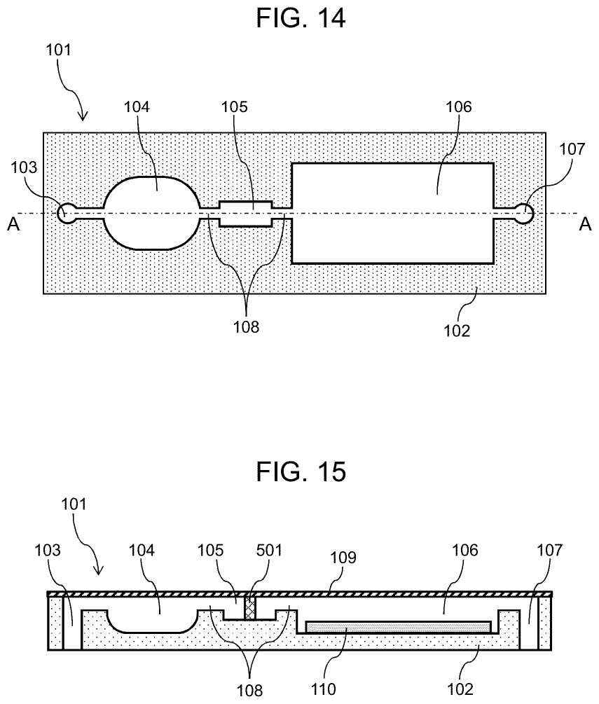

FIG. 14 illustrates a plan view of the fluid device 101. FIG. 15 illustrates a cross-sectional view taken along line A-A of FIG. 14. The present example differs from Example 1 in that in comparison with the configuration illustrated in FIG. 1, the gene amplification section 104 and the metal nanoparticles separating section 105 are provided in different spaces in the fluid device 101 and the metal nanoparticles separating section 105 is provided with a filter 501.

The filter 501 in the metal nanoparticles separating section 105 is used to remove metal nanoparticles from the solution after the plasmonic PCR using the metal nanoparticles is performed in the gene amplification section 104. The filter 501 is, for example, an ultrafiltration membrane. The pore size of the filter 501 is not particularly limited, as long as it is smaller than the shorter side or the diameter of the metal nanoparticle and it allows DNA to pass through, and any known filters can be used. The solution that has passed through the filter 501 and from which the metal nanoparticles are removed is delivered to the gene detection section 106 and undergoes the multiplex gene detection.

The gene amplification section 104, the metal nanoparticles separating section 105, and the gene detection section 106 may be arranged in any order or at any positions in the fluid device 101. The gene amplification section 104, the metal nanoparticles separating section 105, and the gene detection section 106 may not be arranged in this order as illustrated in FIG. 14 and FIG. 15, and for example, the gene amplification section 104 may be positioned between the metal nanoparticles separating section 105 and the gene detection section 106.

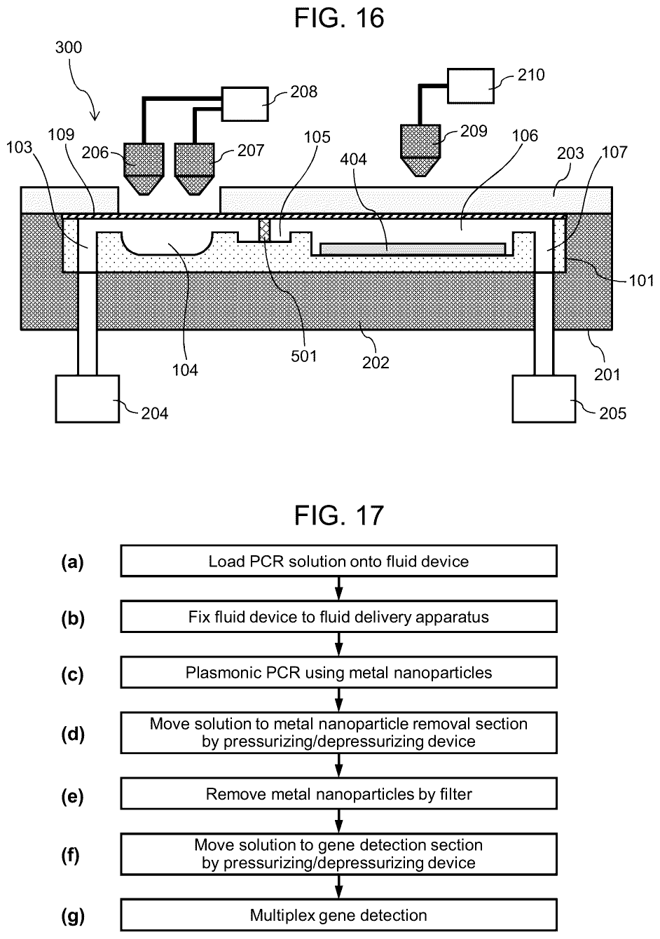

Next, description will be made of the fluid delivery apparatus in which any of the fluid device of FIG. 14 and FIG. 15 and the modifications can be installed. As an example, FIG. 16 illustrates the fluid delivery apparatus 201 in which the fluid device of FIG. 14 and FIG. 15 is installed. The configuration illustrated in FIG. 16 differs from Example 1 in that the external magnetic source 211 is not provided.

The fluid delivery apparatus 201 of Example 2 can be used to deliver a solution in the fluid device 101. Further, the fluid delivery apparatus 201 can provide, to the fluid device 101, light, air pressure, a sensor, and the like that are necessary for performing gene amplification using metal nanoparticles, removal of the metal nanoparticles using the filter 501, and the multiplex gene detection in the fluid device 101.

FIG. 17 illustrates an example of an operation flow of genetic testing using the fluid delivery apparatus 201. The operation flow is for performing the plasmonic PCR using metal nanoparticles, removal of the metal nanoparticles, and the multiplex gene detection. The flow from introducing a solution onto the fluid device 101 to performing the plasmonic PCR using metal nanoparticles (FIG. 17(a) to (c)) is the same as that of Example 1 (FIG. 4(a) to (c)).

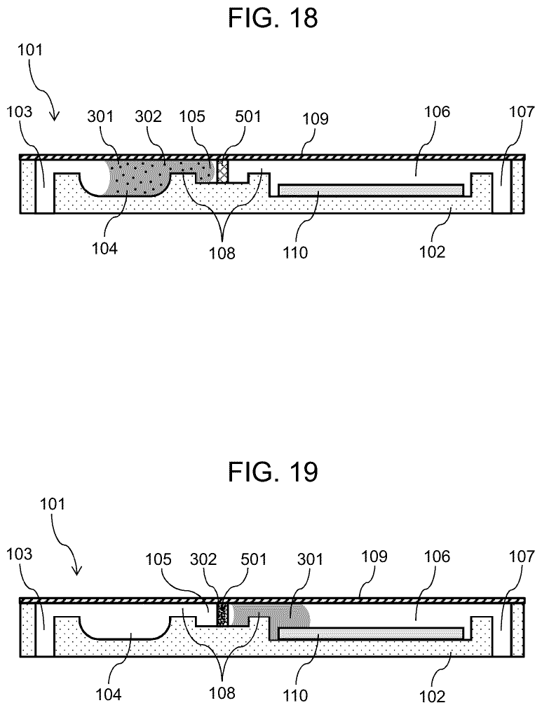

After the plasmonic PCR ends, the solution 301 is delivered toward the metal nanoparticles separating section 105 by means of the pressurizing/depressurizing device 204 (FIG. 17(d)). FIG. 18 schematically illustrates the position of the solution 301 and the state of the metal nanoparticles 302 at this stage. At the stage of FIG. 17(d), the metal nanoparticles 302 are dispersed in the solution 301.

By further continuing the fluid delivery, the removal of the metal nanoparticles 302 by the filter 501 is performed (FIG. 17(e)). FIG. 19 schematically illustrates the position of the solution 301 and the state of the metal nanoparticles 302 at this stage. The metal nanoparticles 302 cannot pass through the filter 501 and are captured on the surface of or inside the filter 501. Meanwhile, the solution 301 containing DNA passes through the filter 501.

By continuing the fluid delivery by means of the pressurizing/depressurizing device 204, the solution 301 from which the metal nanoparticles 302 are removed moves to the gene detection section 106 (FIG. 17(f)). Finally, the gene detection section 106 performs the multiplex gene detection (here, the chromatography) (FIG. 17(g)).

Note that as also described in Example 1, the double-stranded DNA amplified in the PCR may be provided for the gene detection after being resolved into the single-stranded DNA. The resolving into the single-stranded DNA may be performed either before or after the removal of the metal nanoparticles.

### Example 3

Hereinafter, Example 3 will be described. The present example relates to the fluid device and the fluid delivery apparatus for performing gene amplification using metal nanoparticles, removal of metal nanoparticles using a buffer, and multiplex gene detection based on the chromatography method. Note that the descriptions of the portions common to Example 1 or Example 2 will be omitted in some cases.

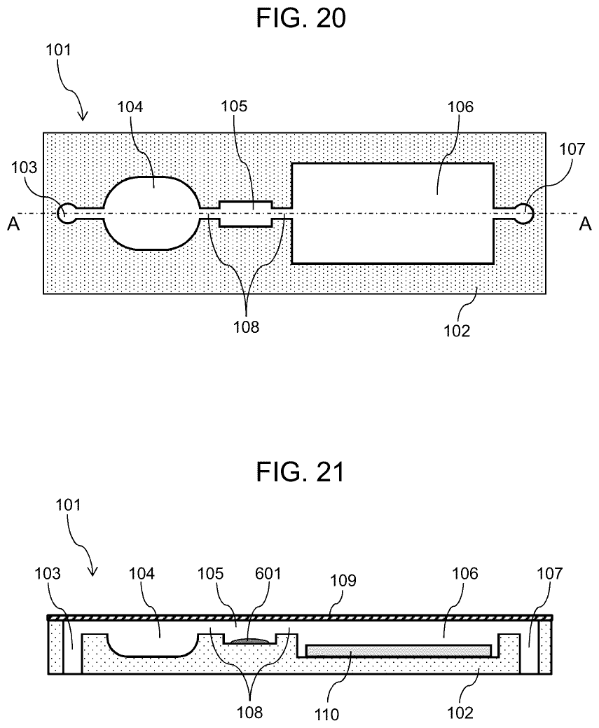

FIG. 20 illustrates a plan view of the fluid device 101. FIG. 21 illustrates a cross-sectional view taken along line A-A of FIG. 20. The present example differs from Example 2 in that in comparison with the configuration illustrated in FIG. 14 and FIG. 15, the metal nanoparticles separating section 105 is provided with a buffer 601 in place of the filter 501.

The buffer 601 in the metal nanoparticles separating section 105 is used to remove metal nanoparticles from the solution after the plasmonic PCR using the metal nanoparticles is performed in the gene amplification section 104. The metal nanoparticles used for the plasmonic PCR are preferably dispersed in the PCR solution to uniformly heat the solution. Thus, to prevent aggregation of the metal nanoparticles, it is common to perform surface modification so that the surfaces of the metal nanoparticles have charges in such a manner as being electrostatically repulsive among the particles.

In the present example, the salt concentration of the solution is changed to cancel out the charges on the surfaces of the metal nanoparticles by the salt and the metal nanoparticles are aggregated, and the buffer 601 to remove the metal nanoparticles from the solution is used. The buffer 601 is, for example, a solution containing sodium chloride. For the buffer 601, any known buffer, without being particularly limited, can be used, as long as it functions to cancel out the charges on the surfaces of the metal nanoparticles.

The buffer 601 may be retained, in advance, in a liquid form in the metal nanoparticles separating section 105. The buffer 601 may also be retained, in advance, in a solid form in the metal nanoparticles separating section 105. In this case, it is only necessary to dissolve the buffer 601 in a solid form with the solution after the PCR.

Alternatively, the buffer 601 may not be retained, in advance, in the metal nanoparticles separating section 105. For example, a chamber storing the buffer 601 in a liquid form may be provided inside or outside the fluid device 101 so as to deliver the buffer 601 from the chamber to the metal nanoparticles separating section 105.

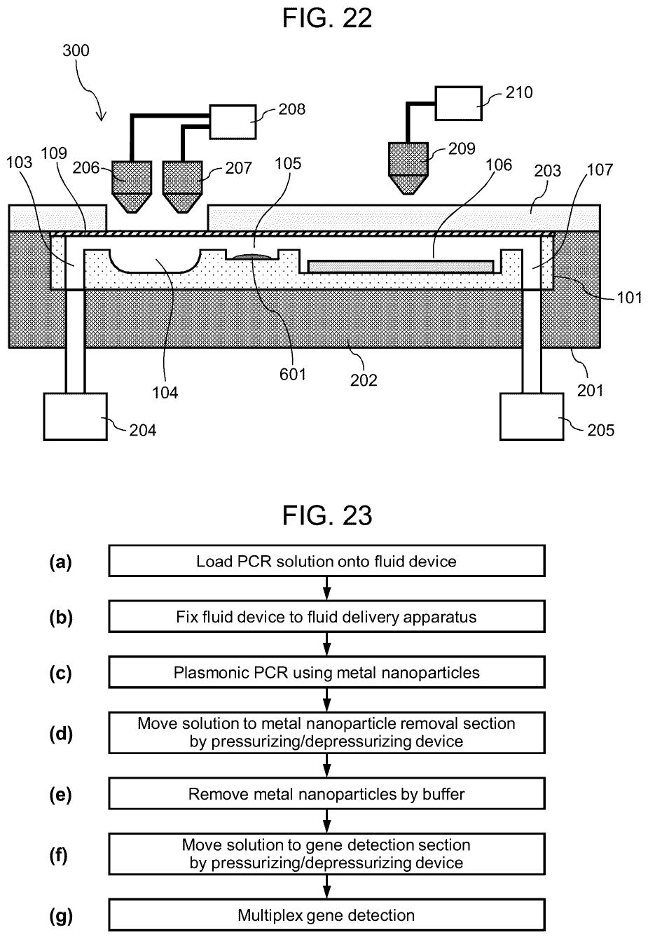

Next, description will be made of the fluid delivery apparatus in which any of the fluid device of FIG. 20 and FIG. 21 and the modifications can be installed. As an example, FIG. 22 illustrates the fluid delivery apparatus 201 in which the fluid device of FIG. 20 and FIG. 21 is installed.

The delivery device 201 of Example 3 can be used to deliver a solution in the fluid device 101. Further, the fluid delivery apparatus 201 can provide, to the fluid device 101, light, air pressure, a sensor, and the like that are necessary for performing gene amplification using metal nanoparticles, removal of the metal nanoparticles using the buffer 601, and the multiplex gene detection in the fluid device 101.

FIG. 23 illustrates an example of an operation flow of genetic testing using the fluid delivery apparatus 201. The operation flow is for performing the plasmonic PCR using metal nanoparticles, removal of the metal nanoparticles, and the multiplex gene detection. The flow from introducing a solution onto the fluid device 101 to delivering the solution 301 to the metal nanoparticles separating section 105 by means of the pressurizing/depressurizing device 204 (FIG. 23(a) to(d)) is the same as that of Example 2 (FIG. 17(a) to (d)).

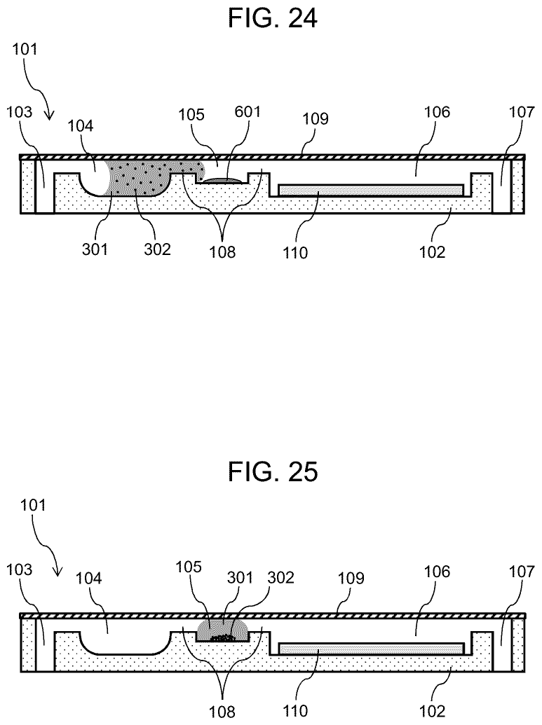

As with FIG. 17 and FIG. 18, at the time when the solution 301 is delivered toward the metal nanoparticles separating section 105 by means of the pressurizing/depressurizing device 204, the metal nanoparticles 302 are dispersed in the solution 301. FIG. 24 schematically illustrates the position of the solution 301 and the state of the metal nanoparticles 302 at this stage.

Further, by continuing the fluid delivery, the buffer 601 in the metal nanoparticles separating section 105, the solution 301, and the metal nanoparticles 302 are mixed together. Thus, the charges on the surfaces of the metal nanoparticles 302 are cancelled out so that the metal nanoparticles 302 aggregate and precipitate (FIG. 23(e)). FIG. 25 schematically illustrates the position of the solution 301 and the state of the metal nanoparticles 302 at this stage.

A supernatant portion without containing the metal nanoparticles is moved from the metal nanoparticles separating section 105 to the gene detection section 106 by means of the pressurizing/depressurizing device 204 (FIG. 23(f)).

Finally, the gene detection section 106 performs the multiplex gene detection (here, the chromatography) (FIG. 23(g)).

Note that as also described in Example 1, the double-stranded DNA amplified in the PCR may be provided for the gene detection after being resolved into the single-stranded DNA. The resolving into the single-stranded DNA may be performed either before or after the removal of the metal nanoparticles.

### Example 4

Hereinafter, Example 4 will be described. The present example relates to the fluid device and the fluid delivery apparatus for performing gene amplification using metal nanoparticles, removal of metal nanoparticles using optical condensation, and multiplex gene detection based on the chromatography method. Note that the descriptions of the portions common to Example 1 or Example 2 will be omitted in some cases.

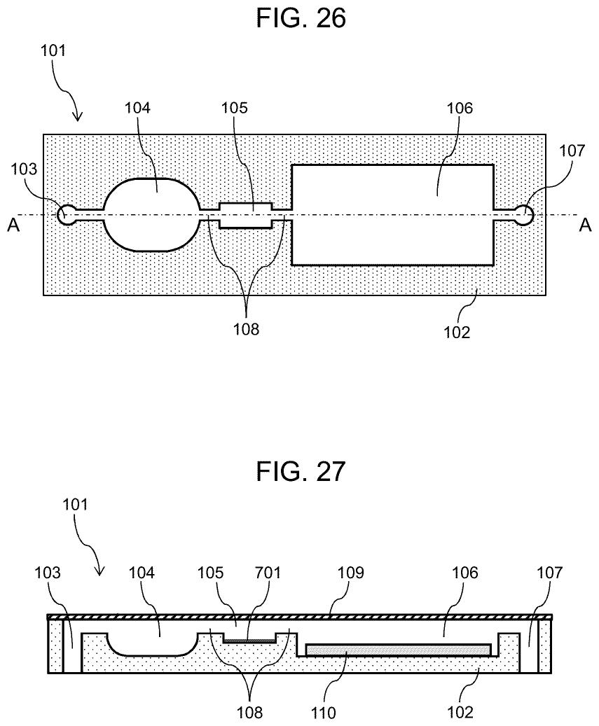

FIG. 26 illustrates a plan view of the fluid device 101. FIG. 27 illustrates a cross- sectional view taken along line A-A of FIG. 26. The present example differs from Example 2 and Example 3 in that in comparison with the configuration illustrated in FIG. 14 and FIG. 15 or FIG. 20 and FIG. 21, the metal nanoparticles separating section 105 is provided with a gold thin film 701 in place of the filter 501 or the buffer 601.

The gold thin film 701 in the metal nanoparticles separating section 105 is used to remove, using a phenomenon called optical condensation, metal nanoparticles from the solution after the plasmonic PCR using the metal nanoparticles is performed in the gene amplification section 104.

When a gold thin film is irradiated with a laser, the light energy is converted into the heat energy by the plasmon resonance of the gold. With the photothermal effect, the temperature of the solution near the gold thin film rapidly rises and microbubbles are generated so as to contact the gold thin film, thereby generating convection around the microbubbles. It is known that fine particles in the solution agitated through convection are condensed around the microbubbles, and this phenomenon is called optical condensation.

In the present example, the gold thin film 701 is provided in the metal nanoparticles separating section 105 to condense and remove the gold nanoparticles in the solution by utilizing the aforementioned phenomenon.

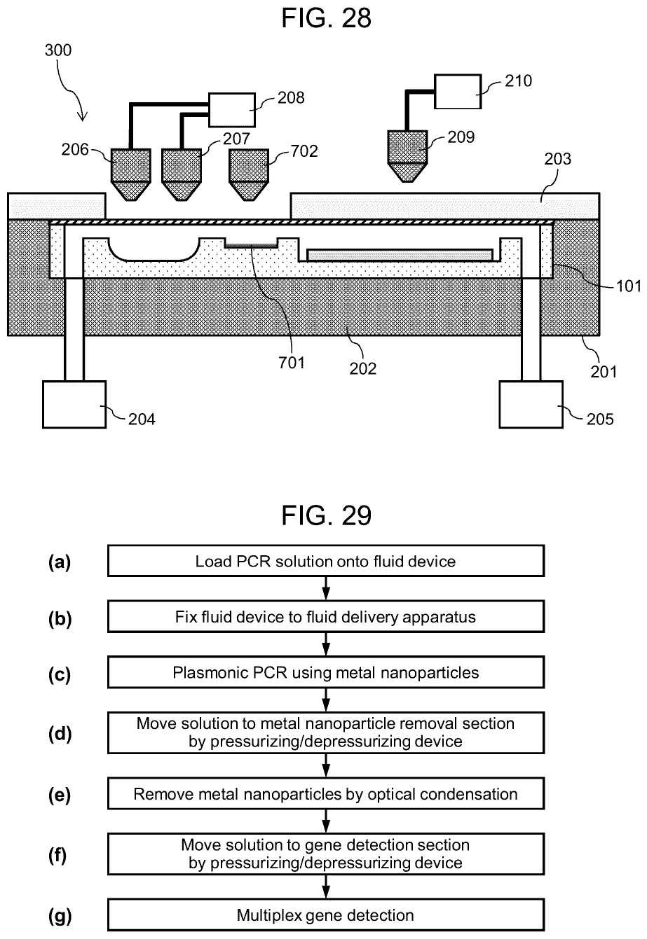

Next, description will be made of the fluid delivery apparatus in which any of the fluid device of FIG. 26 and FIG. 27 and the modifications can be installed. As an example, FIG. 28 illustrates the fluid delivery apparatus 201 in which the fluid device of FIG. 26 and FIG. 27 is installed. The configuration illustrated in FIG. 28 differs from Example 2 and Example 3 in that the metal nanoparticles separating section 105 is provided with the gold thin film 701, in place of the filter 501 or the buffer 601, and a light source 702.

The fluid delivery apparatus 201 of Example 4 can be used to deliver a solution in the fluid device 101. Further, the fluid delivery apparatus 201 can provide, to the fluid device 101, light, air pressure, a sensor, and the like that are necessary for performing gene amplification using metal nanoparticles, removal of the metal nanoparticles using the optical condensation, and the multiplex gene detection in the fluid device 101.

The light source 702 is used to irradiate the metal nanoparticles separating section 105 with light having a specific wavelength so as to induce plasmon resonance of the gold thin film 701. The light source 702, is for example, a laser. For the specific wavelength, a wavelength close to the absorption maximum (reflection minimum) wavelength regarding the surface plasmon resonance of the gold thin film used is selected.

FIG. 28 illustrates an example of an operation flow of genetic testing using the fluid delivery apparatus 201. The operation flow is for performing the plasmonic PCR using metal nanoparticles, removal of the metal nanoparticles, and the multiplex gene detection. The flow from introducing a solution onto the fluid device 101 to delivering the solution 301 to the metal nanoparticles separating section 105 by means of the pressurizing/depressurizing device 204 (FIG. 29(a) to (d)) is the same as that of Example 2 (FIG. 17(a) to (d)) and Example 3 (FIG. 23(a) to (d)).

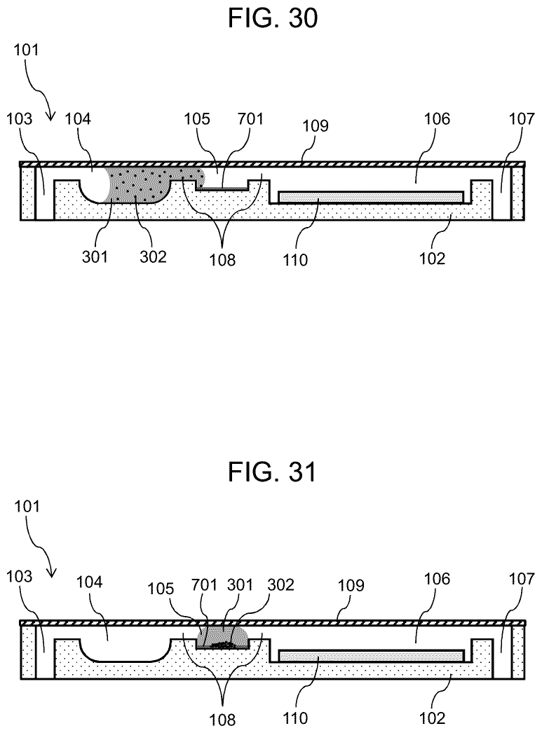

As with Example 2 and Example 3, at the time when the solution 301 is delivered toward the metal nanoparticles separating section 105 by means of the pressurizing/depressurizing device 204, the metal nanoparticles 302 are dispersed in the solution 301. FIG. 30 schematically illustrates the position of the solution 301 and the state of the metal nanoparticles 302 at this stage.

Further, by continuing the fluid delivery, the solution 301 is placed on the gold thin film 701 in the metal nanoparticles separating section 105. The light source 702 is used to irradiate the solution 301 and the gold thin film 701 with light to condense the metal nanoparticles 302 by the optical condensation (FIG. 29(e)). FIG. 31 schematically illustrates the position of the solution 301 and the state of the metal nanoparticles 302 at this stage.

A supernatant portion without containing the metal nanoparticles is moved from the metal nanoparticles separating section 105 to the gene detection section 106 by means of the pressurizing/depressurizing device 204 (FIG. 29(f)). Finally, the gene detection section 106 performs the multiplex gene detection (here, the chromatography) (FIG. 29(g)).

### Modification

Note that the present disclosure is not limited to the aforementioned examples, and includes various modifications. The aforementioned examples are described in detail for easier understanding of the present disclosure, and are not necessarily limited to those including all the described configurations. Further, a part of the configuration of one example can be replaced with the configuration of another example or the configuration of one example can be added to the configuration of another example. Further, for a part of the configuration of each example, addition of and replacement with another configuration and deletion are available.

### DESCRIPTION OF SYMBOLS

101 fluid device
102 substrate
103 solution introducing section
104 gene amplification section
105 metal nanoparticles separating section
106 gene detection section
107 valve connecting section
108 flow path
109 sealing material
110 solid phase carrier section
201 fluid delivery apparatus
202 device retaining section
203 cover
204 pressurizing/depressurizing device
205 valve
206 temperature sensor
207 light source
208 control section
209 sensor
210 signal detection section
211 external magnetic source
281 processor
282 primary memory section
283 auxiliary memory section
284 interface
301 solution
300 fluid delivery system
302 metal nanoparticles
401 well
402 external heat source
403 temperature control section
404 probe spot
405 cleaning solution storage
406 directional valve
501 filter
601 buffer
701 gold thin film
702 light source

What is claimed is:

1. A fluid device comprising:
a gene amplification section that stores a fluid containing metal nanoparticles, the gene amplification section configured to heat the fluid using a photothermal effect of the metal nanoparticles to amplify a gene in the fluid;
a metal nanoparticles separating section configured to separate the metal nanoparticles from the fluid; and
a gene detection section that is a space different from the metal nanoparticles separating section, the gene detection section configured to detect the gene in the fluid from which the metal nanoparticles are separated in the metal nanoparticles separating section.

2. The fluid device according to claim 1, wherein the metal nanoparticles separating section and the gene detection section are connected by means of a flow path.

3. The fluid device according to claim 1, wherein the gene amplification section and the metal nanoparticles separating section are a same space.

4. The fluid device according to claim 1, wherein the metal nanoparticles separating section comprises a filter that separates the gene amplified and the metal nanoparticles.

5. The fluid device according to claim 1, wherein the metal nanoparticles separating section comprises a buffer that causes the metal nanoparticles in the fluid to aggregate by changing a salt concentration of the fluid.

6. The fluid device according to claim 1, wherein the metal nanoparticles separating section comprises a gold thin film that condenses the metal nanoparticles by generating convection in the fluid through light irradiation.

7. The fluid device according to claim 1, wherein the gene detection section is configured to detect the gene amplified, in a multiplex manner, based on position information.

8. The fluid device according to claim 7, wherein the gene detection section comprises a plurality of wells that are assigned position information and in which a real time PCR is performed.

9. The fluid device according to claim 7, wherein the gene detection section comprises a plurality of probe spots that are assigned position information, each probe spot including a probe corresponding to the gene as a detection target.

10. The fluid device according to claim 1, wherein the metal nanoparticles include gold.

11. A fluid delivery system comprising:
a fluid device including:
a gene amplification section that stores a fluid containing metal nanoparticles, the gene amplification section configured to heat the fluid using a photothermal effect of the metal nanoparticles to amplify a gene in the fluid;
a metal nanoparticles separating section configured to separate the metal nanoparticles from the fluid; and
a gene detection section that is a space different from the metal nanoparticles separating section, the gene detection section configured to detect the gene in the fluid from which the metal nanoparticles are separated in the metal nanoparticles separating section; and
a fluid delivery apparatus in which the fluid device can be installed, the fluid delivery apparatus configured to deliver, from the metal nanoparticles separating section to the gene detection section, the fluid from which the metal nanoparticles are separated in the metal nanoparticles separating section.

12. The fluid delivery system according to claim 11, wherein the metal nanoparticles comprise a magnetic substance, the fluid delivery system further comprising:
a magnet and a moving mechanism that brings the magnet into close contact with the metal nanoparticles separating section; or
an electromagnet provided near the metal nanoparticles separating section and an energization control section that controls energization of the electromagnet.

13. A treatment method of a fluid containing metal nanoparticles, the treatment method comprising:
heating a fluid using a photothermal effect of metal nanoparticles contained in the fluid to amplify a gene in the fluid;
separating the metal nanoparticles from the fluid;
delivering, from a first space where the metal nanoparticles are separated to a second space different from the first space, the fluid from which the metal nanoparticles are separated; and
detecting the gene in the fluid in the second space.

14. The treatment method of a fluid containing metal nanoparticles according to claim 13, wherein
the separating the metal nanoparticles includes:
separating the metal nanoparticles containing a magnetic substance from the fluid by bringing a magnet into close contact with the first space; or
separating the metal nanoparticles containing the magnetic substance from the fluid by energizing an electromagnet provided near the first space.

15. The treatment method of a fluid containing metal nanoparticles according to claim 14, wherein the delivering, to the second space, the fluid from which the metal nanoparticles are separated includes delivering, to the second space, the fluid from which the metal nanoparticles are separated, with the magnet kept close contact with the first space or with the electromagnet energized.
